(19) United States
(12) Patent Application Publication
(10) Pub. No.: US 2011/0052580 A1
(43) Pub. Date: Mar. 3, 2011

(54) USE OF PICOPLATIN AND BEVACIZUMAB TO TREAT COLORECTAL CANCER

(75) Inventors: Ronald A. Martell, San Francisco, CA (US); David A. Karlin, Los Altos, CA (US)

(73) Assignee: Poniard Pharmaceuticals, Inc., Seattle, WA (US)

(21) Appl. No.: 12/866,702

(22) PCT Filed: Feb. 6, 2009
(86) PCT No.: PCT/US09/00770

§ 371 Date: Nov. 11, 2010

Related U.S. Application Data
(60) Provisional application No. 61/027,360, filed on Feb. 8, 2008.
(60) Provisional application No. 61/027,382, filed on Feb. 8, 2008.
(60) Provisional application No. 61/027,387, filed on Feb. 8, 2008.

(51) Int. Cl. A61K 39/395; A61P 35/00; A61P 35/04

(52) U.S. Cl. 424/133.1

(57) ABSTRACT

The invention provides a method of treatment of metastatic colorectal cancer by administration of the anti-cancer platinum drug picoplatin in conjunction with bevacizumab (Avastin®) and optionally, with 5-FU and leucovorin in a variety of treatment regimens. The invention also provides a use of picoplatin in conjunction with bevacizumab and, optionally, 5-FU and leucovorin, for the treatment of metastatic colorectal cancer.

### CROSS REFERENCE TO RELATED APPLICATION

This application claims the priority of U.S. Ser. No. 61/027,387, filed Feb. 8, 2008, U.S. Ser. No. 61/027,382, filed Feb. 8, 2008, and U.S. Ser. No. 61/027,360, filed Feb. 8, 2008, the disclosures of which are incorporated by reference herein in their entireties. This application also claims the priority of U.S. Ser. Nos. 60/857,066 (filed Nov. 6, 2006), 60/857,725 (filed Nov. 8, 2006), 60/877,495 (filed Dec. 28, 2006), 60/889,191 (filed Feb. 9, 2007), 60/931,589 (filed May 24, 2007), and 60/983,852 (filed Oct. 30, 2007), and of U.S. Ser. No. 11/982,841, filed Nov. 5, 2007, the disclosures of which are incorporated by reference herein in their entireties.

### BACKGROUND

Colorectal cancer remains the second most common cause of cancer-related death in the United States and a significant cause of cancer-related death in other countries as well.¹ For decades, the only approved chemotherapeutic drug for treatment of metastatic colorectal cancer (MCRC) was 5-fluorouracil (5-FU), and it continues to be the backbone of most first-line chemotherapeutic regimens for patients with advanced disease. However, there has been much progress made in treatment of colorectal cancer in the past decade, with the approval of several new therapeutic agents including irinotecan, oxaliplatin, capecitabine, and most recently, cetuximab and bevacizumab.^{2,3} Importantly, a variety of new chemotherapeutic regimens utilizing these agents have been devised, which have led to increased response rates and incremental increases in the time to progression and median survival for patients with advanced disease.^{2,3} Response rates for 5-FU/leucovorin, irinotecan, and oxaliplatin as single agent therapy have been low (23%, 18%, and 12%, respectively), progression-free survival has been short (median 4.0, 4.3, and 4.0 months, respectively), and median survival has also been short, approximately (12, 12, and 14.5 months, respectively).⁴ With the introduction of 5-FU-based combination chemotherapeutic regimens using irinotecan and oxaliplatin, “FOLFOX regimens,” the response rate has increased substantially, with response rates reported as high as 64% (FOLFOX7), time to progression ranging from 8.9-12.3 months, and median survival now approaching approximately 20 months in some reports.²⁻⁴

Avastin® (bevacizumab) is a recombinant humanized monoclonal IgGl antibody that binds to and inhibits the biologic activity of human vascular endothelial growth factor (VEGF) as shown by in vitro and in vivo assay systems. Bevacizumab binds VEGF and prevents the interaction of VEGF to its receptors (Flt-1 and KDR) on the surface of endothelial cells. The interaction of VEGF with its receptors leads to endothelial cell proliferation and new blood vessel formation in in vitro models of angiogenesis. Administration of bevacizumab to xenotransplant models of colon cancer in nude (athymic) mice caused reduction of microvascular growth and inhibition of metastatic disease progression.

Bevacizumab contains human framework regions and the complementarity-determining regions of a murine antibody that binds to VEGF (Presta L G, Chen H, O'Connor S J, Chisholm V, Meng Y G, Krummen L, et al. Humanization of an anti-vascular endothelial growth factor monoclonal antibody for the therapy of solid tumors and other disorders. Cancer Res 1997; 57:4593-9). Bevacizumab is produced in a Chinese Hamster Ovary mammalian cell expression system in a nutrient medium containing the antibiotic gentamicin and has a molecular weight of approximately 149 kilodaltons.

Cisplatin, the first platinum analogue, was introduced approximately 20 years ago and is still widely used. The approval of cisplatin was followed by approval of carboplatin, and most recently by that of oxaliplatin.

Treatment with platinum analogues is limited by their toxicity. While neurotoxicity and nephrotoxicity are the main dose-limiting toxicities (DLT) observed following cisplatin treatment, myelosuppression is most significant following carboplatin treatment. Carboplatin is known to cause cumulative dose-related toxicity that results in slow bone marrow recovery. Peripheral neurotoxicity is well documented in patients treated with oxaliplatin.

Regimens containing irinotecan are associated with significant diarrhea and other gastrointestinal toxicity, while those containing oxaliplatin are associated with neurotoxicity.²⁻¹⁰° The neurotoxicity observed is of two types: first, a cumulative and often dose limiting sensory loss with paresthesias that can interfere with function and second, a disturbing cold sensitivity that limits patient acceptance of the FOLFOX regimen.⁷⁻¹⁰ The efficacy of platinum analogues is also limited by several (intrinsic or acquired) mechanisms of resistance, including impaired cellular uptake, intracellular inactivation by thiols [e.g., reduced glutathione], and enhanced DNA repair and/or increased tolerance to platinum-DNA adducts.²³ Pre-clinical studies indicate that picoplatin can overcome these three mechanisms of resistance. This has been demonstrated in vitro and by using human ovarian xenograft tumor models that exhibit resistance to cisplatin.¹³⁻¹⁷

### SUMMARY

The present invention is directed to methods of treatment of colorectal cancer with picoplatin, bevacizumab (Avastin®) and, optionally, 5-fluorouracil and/or leucovorin; and to uses of picoplatin in conjunction with bevacizumab and, optionally, 5-fluorouracil and/or leucovorin, to treat metastatic colorectal cancer.

In various embodiments, the invention provides a method of treatment of colorectal cancer, comprising administering to a patient afflicted with colorectal cancer picoplatin, bevacizumab, 5-fluorouracil (5-FU) and leucovorin, wherein 5-FU and leucovorin are administered intravenously at least twice at intervals of about 2-6 weeks, the picoplatin is administered with the leucovorin and 5-FU every other time that the fluorouracil and leucovorin are administered, and the bevacizumab is administered at least twice at one-week intervals. For example, the picoplatin can be administered at a dose of about 60-180 mg/m², preferably at a dose of about 150 mg/m². For example, the interval of administration of the 5-FU and the leucovorin can be about two weeks and the interval of administration of the picoplatin can be about four weeks.

In various embodiments, the invention provides a method of treatment of colorectal cancer, comprising administering to a patient afflicted with colorectal cancer effective amounts of a combination of picoplatin, bevacizumab, 5-FU and leucovorin, wherein the picoplatin, and the 5-FU and the leucovorin are administered intravenously at least twice at intervals of about 2-6 weeks, and the bevacizumab is administered at least twice at one-week intervals, wherein an amount of picoplatin administered is less than the maximum tolerated dose of picoplatin. For example, the picoplatin can be administered at a dose of about 45-150 mg/m², preferably at a dose of about 135-150 mg/m². For example, the interval of administration of the picoplatin, 5-FU and the leucovorin can be about two weeks.

Another embodiment of the invention provides a method of treatment of colorectal cancer, comprising administering to a patient afflicted with metastatic colorectal cancer picoplatin, bevacizumab, 5-FU, and leucovorin, wherein 5-FU and leucovorin are administered intravenously at intervals of about two weeks, and the picoplatin is administered with the leucovorin and 5-FU every time that the fluorouracil and leucovorin are administered, wherein the picoplatin is administered at a dose of about 45-120 mg/m², and wherein the bevacizumab is administered intravenously at a dose of about 5-25 mg/kg, preferably at 10 mg/kg, at biweekly intervals.

In another embodiment of the invention, the picoplatin is administered substantially concurrently with the leucovorin and the picoplatin is administered at every second treatment of the patient with the 5-FU and the leucovorin, e.g., every four weeks. The bevacizumab is administered concurrently with the picoplatin and then biweekly thereafter. The leucovorin can be administered at a dosage of about 200-500 mg/m², preferably at about 400 mg/m². The picoplatin is administered at a dosage of about 60-180 mg/m². The bevacizumab, formulated as the solution Avastin®, described above, is administered by infusion at a dose of 10 mg/kg every 14 days. The 5-FU is administered at a total dosage of about 1000-3000 mg/m². A preferred treatment cycle for leucovorin and 5-FU is every two weeks, and picoplatin is administered every 4 weeks, e.g., at a low dose of about 60-75 mg/m², e.g., 60 mg/m², or at a high dose of about 120-180 mg/m², preferably about 120-150 mg/m², e.g. about 150 mg/m².

The present invention also provides a method further comprising administering the picoplatin in a dosage form comprising an isotonic solution comprising water, a tonicity adjuster, and about 0.5 mg/mL dissolved picoplatin. The dosage form can also comprise an effective amount of dissolved or dispersed 5-FU and/or leucovorin in accord with the doses disclosed herein. The dosage form also does not contain a preservative or bacteriostatic agent. An appropriate volume of the dosage form can be administered to achieve a desired therapeutic dose.

The dosage form also can comprise a first container comprising the picoplatin solution and a second container comprising a solution of bevacizumab. The two containers can further comprise means to simultaneously administer the contents to a patient, e.g., the containers can be plastic intravenous bags that can be independently connected to a single intravenous tube so that the content of each container can be simultaneously administered to the patient, e.g., via a Y-link. These containers can be packaged together with instructions regarding their end-use, e.g., in a kit. A separately packaged leucovorin solution, and/or a separately packed 5-FU solution, can also be included in the kit. The picoplatin solution can be a dosage form of picoplatin at a concentration of about 0.5 mg/mL, optionally comprising a tonicity adjuster such as NaCl, wherein no preservative of bacteriostatic agent is present in the dosage form.

In an embodiment, the invention provides a use of picoplatin in conjunction with bevacizumab, 5-fluorouracil (5-FU), and leucovorin to treat metastatic colorectal cancer, wherein the 5-FU and leucovorin are administered intravenously at least twice at intervals of about 2-6 weeks, the picoplatin is administered with the leucovorin and 5-FU every other time that the fluorouracil and leucovorin are administered, and the bevacizumab is administered at least twice at two-week intervals.

In an embodiment, the invention provides a use of picoplatin in conjunction with bevacizumab, 5-fluorouracil (5-FU), and leucovorin to treat metastatic colorectal cancer, wherein the picoplatin, 5-FU and leucovorin are administered intravenously at least twice at intervals of about two weeks, and the bevacizumab is administered at least twice at two-week intervals, wherein the amount of picoplatin is less than the maximum tolerated dose of picoplatin when administered in said combination.

In an embodiment, the invention provides a use of picoplatin in conjunction with bevacizumab, 5-fluorouracil (5-FU), and leucovorin to treat metastatic colorectal cancer, wherein 5-FU and leucovorin are administered intravenously at intervals of about two weeks, and the picoplatin is administered with the leucovorin and 5-FU every time that the fluorouracil and leucovorin are administered, wherein the picoplatin is administered at a dose of about 45-120 mg/m², and wherein the bevacizumab is administered intravenously at a dose of 5-25 mg/kg at biweekly intervals.

In an embodiment, the invention provides a use of about 5-150 mg/m² picoplatin administered about every 21 days in conjunction with a dose of about 10 mg/kg bevacizumab administered about every other week to treat metastatic colorectal cancer in a patient afflicted with colorectal cancer who has failed irinotecan, FOLFOX and/or FOLPI regimens.

In an embodiment, the invention provides a use of about 5-150 mg/m² picoplatin administered about every 21 days in conjunction with a dose of about 10 mg/kg bevacizumab administered about every other week to treat metastatic colorectal cancer in a patient afflicted with colorectal cancer who has received irinotecan, FOLFOX and/or FOLPI regimens with or without bevacizumab or cetuximab to prevent recurrence wherein the cancer is in remission.

### DETAILED DESCRIPTION OF THE INVENTION

In an embodiment, the invention provides a method of treatment of colorectal cancer, comprising administering to a patient afflicted with metastatic colorectal cancer picoplatin, bevacizumab, 5-fluorouracil (5-FU), and leucovorin, wherein 5-FU and leucovorin are administered intravenously at least twice at intervals of about 2-6 weeks, the picoplatin is administered with the leucovorin and 5-FU every other time that the fluorouracil and leucovorin are administered, and the bevacizumab is administered at least twice at two-week intervals.

Another embodiment of the invention provides a method of treatment of colorectal cancer, comprising administering to a patient afflicted with metastatic colorectal cancer effective amounts of a combination of picoplatin, bevacizumab, 5-FU and leucovorin, wherein the picoplatin, 5-FU and leucovorin are administered intravenously at least twice at intervals of about two weeks, and the bevacizumab is administered at least twice at two-week intervals (biweekly), wherein the amount of picoplatin is less than the maximum tolerated dose of picoplatin when administered in said combination.

Another embodiment of the invention provides a method of treatment of colorectal cancer, comprising administering to a patient afflicted with metastatic colorectal cancer picoplatin, bevacizumab, 5-FU, and leucovorin, wherein 5-FU and leucovorin are administered intravenously at intervals of about two weeks, and the picoplatin is administered with the leucovorin and 5-FU every time that the fluorouracil and leucovorin are administered, wherein the picoplatin is administered at a dose of about 45-120 mg/m², and wherein the bevacizumab is administered intravenously at a dose of about 5-25 mg/kg, preferably at 10 mg/kg, at biweekly intervals.

In another embodiment of the invention, the picoplatin is administered substantially concurrently with the leucovorin and the picoplatin is administered at every second treatment of the patient with the 5-FU and the leucovorin, e.g., every four weeks. The bevacizumab is administered concurrently with the picoplatin and then biweekly thereafter. The leucovorin can be administered at a dosage of about 200-500 mg/m², preferably at about 400 mg/m². The picoplatin is administered at a dosage of about 60-180 mg/m². The bevacizumab, formulated as the solution Avastin®, described above, is administered by infusion at a dose of 10 mg/kg every 14 days. The 5-FU is administered at a total dosage of about 1000-3000 mg/m². A preferred treatment cycle for leucovorin and 5-FU is every two weeks, and picoplatin is administered every 4 weeks, e.g., at a low dose of about 60-75 mg/m², e.g., 60 mg/m², or at a high dose of about 120-180 mg/m², preferably about 120-150 mg/m², e.g. about 150 mg/m².

Therefore, in one embodiment of the invention, the leucovorin, at a dosage of 200-500 mg/m², is administered as an about 2 hour infusion concurrently with the picoplatin, when it is given, wherein the picoplatin dosage is 120-180 mg/m², e.g., about 150 mg/m²; the administration of the leucovorin and the picoplatin being followed by a 5-FU dosage of about 400 mg/m² as a bolus; the 5-FU dosage being followed by 5-FU at a dosage of 600 mg/m² or 2,400 mg/m², preferably administered as a 22 hour or as a 46 hour continuous infusion, respectively, wherein the leucovorin and 5-FU are provided to the patient at intervals of two weeks and the leucovorin, picoplatin, and 5-FU are provided to the patient at alternating intervals of four weeks.

The bevacizumab is administered as described above, at an initial dose of 10 mg/kg followed by biweekly doses of 10 mg/kg. In another embodiment, a low dose of picoplatin of about 45-75 mg/m², e.g., about 60-75 mg/m², e.g., about 60 mg/m², is administered. Such 5-FU/leucovorin/picoplatin regimens can be broadly termed FOLPI regimens which, in the present invention, are supplemented by bevacizumab infusions.

In another embodiment of the invention, the leucovorin, at a dosage of 400 mg/m², is administered as a 2 hour infusion; the administration of the leucovorin being followed by a 5-FU bolus at a dosage of 400 mg/m²; the 5-FU bolus dosage being followed by parenteral 5-FU at a dosage of 400 mg/m² or 2,400 mg/m², preferably administered as a 22 hour or as a 46 hour continuous infusion, respectively; the administration of the leucovorin and the 5-FU taking place every two weeks; wherein every two weeks picoplatin, at a dosage of up to about 50 mg/m², e.g., at about 40-50 mg/m², e.g., about 45 mg/m², is administered concurrently with the leucovorin, preferably simultaneously. Picoplatin dosages of about 45-105 mg/m² can also be administered. Bevacizumab is given weekly as described hereinabove.

It has unexpectedly been found that, in some cases, the combination of low doses of picoplatin administered with leucovorin and 5-FU at every treatment cycle, are as effective as, or more effective than, higher doses, e.g., the maximum tolerated dose (MTD), given at the same intervals, in producing a response. The MTD for the 2 week and 4 week picoplatin administration schedules are discussed below. Preferably, such doses in the initial treatment are lower or substantially lower than the MTD. Such doses can range from about 40-60 mg/m² of picoplatin every two weeks, given with leucovorin and bevacizumab and followed by 5-FU, as discussed below.

It has surprisingly been found that a total cumulative picoplatin dose in excess of about 900 mg/m² can be tolerated by patients without neuropathy of Grade 2 or higher being observed.

In one embodiment of the present method, the patient preferably has not previously had systemic treatment, such as chemotherapy, for metastatic disease. The patient may have, however, received earlier adjuvant therapy at the time of primary tumor treatment, at least 6 months prior to the present picoplatin-bevacizumab treatment.

In an embodiment, the invention provides use of picoplatin in conjunction with bevacizumab, 5-fluorouracil (5-FU), and leucovorin to treat metastatic colorectal cancer, wherein the 5-FU and leucovorin are administered intravenously at least twice at intervals of about 2-6 weeks, the picoplatin is administered with the leucovorin and 5-FU every other time that the fluorouracil and leucovorin are administered, and the bevacizumab is administered at least twice at two-week intervals.

In an embodiment, the invention provides use of picoplatin in conjunction with bevacizumab, 5-fluorouracil (5-FU), and leucovorin to treat metastatic colorectal cancer, wherein the picoplatin, 5-FU and leucovorin are administered intravenously at least twice at intervals of about two weeks, and the bevacizumab is administered at least twice at two-week intervals, wherein the amount of picoplatin is less than the maximum tolerated dose of picoplatin when administered in said combination.

In an embodiment, the invention provides use of picoplatin in conjunction with bevacizumab, 5-fluorouracil (5-FU), and leucovorin to treat metastatic colorectal cancer, wherein 5-FU and leucovorin are administered intravenously at intervals of about two weeks, and the picoplatin is administered with the leucovorin and 5-FU every time that the fluorouracil and leucovorin are administered, wherein the picoplatin is administered at a dose of about 45-120 mg/m², and wherein the bevacizumab is administered intravenously at a dose of 5-25 mg/kg at biweekly intervals.

The use can be a use wherein the patient having metastatic colorectal cancer has not previously been treated for metastatic disease.

Or, the use can be a use wherein the patient having metastatic colorectal cancer has previously been treated with an irinotecan regimen, a FOLFOX regimen, or a FOLPI regimen, wherein the cancer is refractory or wherein the cancer is progressive within six months of completing the regimen.

Or, the use can be a use wherein the patient having metastatic colorectal cancer has previously been treated successively with at least two of the group consisting of an irinotecan regimen, a FOLFOX regimen and a FOLPI regimen wherein the cancer is refractory or wherein the cancer is progressive within six months of completing the regimen. The irinotecan regimen or the FOLFOX regimen or both can have been accompanied by bevacizumab administration.

In various embodiments of the present method, the patient has not previously been treated for metastatic disease, or the patient has not previously had systemic treatment, such as chemotherapy, for localized or metastatic disease. For example, the patient may have had surgery to remove or to de-bulk the primary tumor and then be treated with one of the picoplatin, 5-FU, leucovorin regimens (e.g., FOLPI) of the invention to prevent or delay progression of the cancer, including to prevent or delay the development of metastases. The patient may have received earlier chemotherapy at the time of primary tumor treatment, at least 6 months prior to the present picoplatin treatment.

In various embodiments, the picoplatin can be administered with curative intent, rather than merely seeking to arrest the disease with no remission. The dosage of the picoplatin can be increased beyond that bringing about disease stasis in order to achieve a cure in the patient.

In various embodiments, the picoplatin and the leucovorin can be administered simultaneously.

In various embodiments, the 5-FU can be administered following the administration of the picoplatin, leucovorin and bevacizumab.

In various embodiment, the leucovorin and the 5-FU can be administered about every two weeks, the picoplatin administered with the leucovorin about every 4 weeks, and the bevacizumab administered biweekly.

In various embodiments, the picoplatin can be administered substantially concurrently with the leucovorin followed by administration of the 5-FU at every treatment of the patient, and the bevacizumab is administered at two week intervals.

In various embodiments, the leucovorin can be administered at an initial dosage of about 200-400 mg/m².

In various embodiments, the 5-FU can be administered at a total dosage per dosing of about 1000-3000 mg/m².

In various embodiments, the picoplatin can be administered at a dosage of about 60-180 mg/m², or the picoplatin is administered at a dosage of about 120-180 mg/m².

In various embodiment, a subsequent dose of picoplatin can be administered at about a 15-30 mg/m² lower dose than a previous dose.

In various embodiments, the picoplatin can be administered at least once at a dosage of about 150 mg/m², or the picoplatin can be administered at least once at a dosage of about 60-75 mg/m², or the picoplatin can be administered at least once at a dosage of about 40-45 mg/m².

In various embodiments, a cumulative dose of greater than about 900 mg/m² of picoplatin can be delivered to the patient.

In various embodiments, the bevacizumab can be administered intravenously at a first dose of about 10 mg/kg, then every other week at a dose of about 10 mg/kg.

In various embodiments, the leucovorin, at a dosage of about 400 mg/m², can be administered as a 2 hour infusion, the administration of the leucovorin being followed by a 5-FU bolus at a dosage of about 400 mg/m²; the 5-FU bolus being followed by 5-FU at a dosage of about 2,400 mg/m² administered as a 46 hour continuous infusion; wherein the leucovorin and the 5-FU are administered to the patient every 2 weeks and about 60-150 mg/m² of the picoplatin is administered to the patient with the leucovorin every 4 weeks, wherein at least the initial dose of picoplatin is about 150 mg/m², and wherein the bevacizumab is administered at an initial dose of about 10 mg/kg, then once every other week at a dose of about 10 mg/kg.

In various embodiments, in invention provides a use of about 5-150 mg/m² picoplatin administered about every 21 days in conjunction with a dose of about 10 mg/kg bevacizumab administered about every other week to treat metastatic colorectal cancer in a patient afflicted with colorectal cancer who has failed irinotecan, FOLFOX and/or FOLPI regimens. The 5-FU or leucovorin or both can be administered every other week. In various embodiments, the inventive use can further comprise use of a 5-HT₃ receptor antagonist.

In various embodiments, the invention provides a use of about 5-150 mg/m² picoplatin administered about every 21 days in conjunction with a dose of about 10 mg/kg bevacizumab administered about every other week to treat metastatic colorectal cancer in a patient afflicted with colorectal cancer who has received irinotecan, FOLFOX and/or FOLPI regimens with or without bevacizumab or cetuximab to prevent recurrence wherein the cancer is in remission. The 5-FU or leucovorin or both can be administered every other week. In various embodiments, the inventive use can further comprise use of a 5-HT₃ receptor antagonist.

Picoplatin is a third generation platinum analogue that has demonstrated synergy with 5-FU in vitro in pre-clinical studies and has undergone extensive Phase 1 and 2 testing in a variety of cancers.¹¹⁻²² Like other platinum analogues, picoplatin causes cell death by the formation of covalent cross-links in DNA that interfere with DNA replication and transcription, leading to cell death. The unacceptable nephrotoxicity, oto-, and neurotoxicity associated with earlier platinum analogues has not been reported with picoplatin either in animal studies or in clinical trials.^{11, 19-22} Several human ovarian and colon cell lines with induced resistance to oxaliplatin retain sensitivity to picoplatin.¹⁶⁻¹⁸

In Phase 1 studies with picoplatin, tolerable side-effects and indications of activity were seen in subjects with ovarian cancer, non-small cell lung cancer (NSCLC), small cell lung cancer (SCLC), colorectal cancer, head and neck cancer, renal cell cancer, thymic cancer, pancreatic cancer, stomach cancer, leiomyosarcoma, liver cancer, mesothelioma, and prostate cancers.^{24,25} In Phase 2 studies, indications of efficacy were seen in subjects with ovarian, NSCLC, SCLC, mesothelioma, prostate cancer, and breast cancer.

Picoplatin (SP-4-3) (cis-aminedichloro(2-methylpyridine)Pt(II)), and useful prodrugs and analogs thereof are disclosed in U.S. Pat. Nos. 5,665,771; 6,518,428; 6,413,953; U.S. patent application Ser. No. 11/982,891, filed Nov. 5, 2007; and PCT/GB/01/02060, which are incorporated herein by reference. The doses disclosed herein can be provided by oral administration of an effective amount of picoplatin in combination with a pharmaceutically acceptable vehicle, as well as by intravenous infusion.

Avastin® (bevacizumab) is a recombinant humanized monoclonal IgGl antibody that binds to and inhibits the biologic activity of human vascular endothelial growth factor (VEGF) in in vitro and in vivo assay systems. Bevacizumab is produced in a Chinese Hamster Ovary mammalian cell expression system in a nutrient medium containing the antibiotic gentamicin and has a molecular weight of approximately 149 kilodaltons.

Avastin® is a clear to slightly opalescent, colorless to pale brown, sterile, pH 6.2 solution for intravenous (IV) infusion. Avastin® is supplied in 100 mg and 400 mg preservative-free, single-use vials to deliver 4 mL or 16 mL of Avastin® (25 mg/mL). The 100 mg product is formulated in 240 mg α,α-trehalose dihydrate, 23.2 mg sodium phosphate (monobasic, monohydrate), 4.8 mg sodium phosphate (dibasic, anhydrous), 1.6 mg polysorbate 20, and Water for Injection, USP. The 400 mg product is formulated in 960 mg α,α-trehalose dihydrate, 92.8 mg sodium phosphate (monobasic, monohydrate), 19.2 mg sodium phosphate (dibasic, anhydrous), 6.4 mg polysorbate 20, and Water for Injection, USP. The recommended dose regimen is 10 mg/kg administered every two weeks in combination with a FOLFOX regimen (oxaliplatin, leucovorin (LV), and 5-fluorouracil (5-FU)).

Bevacizumab, in combination with intravenous 5-fluorouracil-based chemotherapy, is presently indicated for first- or second-line treatment of patients with metastatic carcinoma of the colon or rectum (MCRC). In treatment of MCRC, the recommended dose of bevacizumab, used in combination with intravenous 5-FU-based chemotherapy, is administered as an intravenous infusion (5 mg/kg or 10 mg/kg) every 14 days. The recommended dose of bevacizumab, when used in combination with FOLFOX4 for treatment of metastatic colorectal cancer (MCRC) is 10 mg/kg biweekly (14 days) (http://www.gene.com/gene/products/information/oncology/avastin/insert.jsp#ad ministration).

Bevacizumab has been evaluated for the treatment of MCRC (see http://www.gene.com/gene/products/information/oncology/avastin/insert.jsp) in combination with the organoplatinum drug oxaliplatin and with the polycyclic alkaloid derivative irinotecan. In one clinical trial with oxaliplatin, patients received bevacizumab in combination with 5-fluorouracil (5-FU) and leucovorin (LV) in combination with oxaliplatin (85 mg/m²) (FOLFOX4 regimen) versus FOLFOX4 alone as a second-line treatment following irinotecan/5-FU first-line therapy. In the bevacizumab-treated group, overall survival (OS) was significantly longer in patients receiving Avastin® in combination with FOLFOX4 as compared to those receiving FOLFOX4 alone (median OS13.0 mos vs. 10.8 mos; hazard ratio 0.75 [95% CI 0.63, 0.89], p=0.001 stratified logrank test). In addition, patients treated with Avastin® in combination with FOLFOX4 were reported to have significantly longer progression-free survival and a higher overall response rate based on investigator assessment.

The FOLFOX regimens commonly in use, FOLFOX4, FOLFOX6, and FOLFOX7 all combine the same bioactive agents, but at different dosages, as shown in Table 1.

TABLE 1
Summary of FOLFOX Regimens

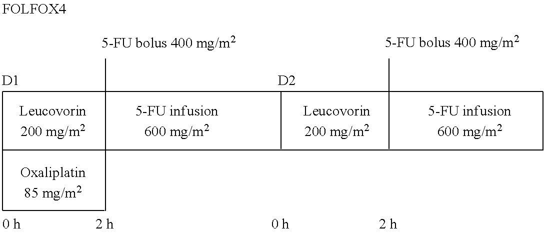

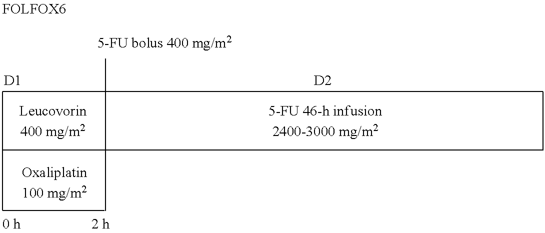

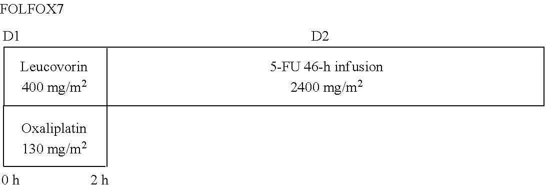

The use of picoplatin replacing oxaliplatin in a FOLFOX regimen, termed a FOLPI regimen, and then a study evaluating the FOLPI regimen with and without concurrent administration of bevacizumab to treat metastatic colorectal cancer will be conducted in three parts. Phase 1 is a dose escalation study to identify the maximum tolerated dose (MTD) of picoplatin that can be administered either every two weeks or every four weeks, with 5-FU and leucovorin (LV) administered every two weeks, as initial therapy for subjects with metastatic colorectal cancer who have not been previously treated for metastatic disease. Phase 2 is a randomized study. In one arm of the study, picoplatin is administered at 150 mg/m² every four weeks, combined with 5-FU and leucovorin that are administered every two weeks (FOLPI). In the other arm, a modified FOLFOX 6 regimen is employed wherein the 100 mg/m² oxaliplatin dose in FOLFOX 6 has been reduced to 85 mg/m², and is administered every 2 weeks, so that the two agents can be compared in the context of a widely used regimen. It is believed that cancer patients can be more effectively treated with the regimens of the present invention, which employ picoplatin instead of cisplatin, carboplatin or oxaliplatin, because they will experience fewer side effects, such as neuropathy, while preferably receiving higher doses of the platinum drug. Phase 3 will be a study comparing the FOLPI regimen with and without biweekly Avastin® infusions.

Subjects eligible for the Phase 1 study will have Stage 1V colorectal cancer and will have received no systemic therapy for metastatic cancer. Prior adjuvant chemotherapy with a 5-FU-based treatment regimen not containing oxaliplatin or irinotecan is acceptable if there has been a treatment-free interval of at least 6 months.

### Phase 1

Subjects are assigned centrally to treatment with picoplatin administered either every two or every four weeks and are assigned a dose of picoplatin to be given dependent on the study results to date. Each patient also receives 5-FU and leucovorin therapy every two weeks. Cohorts of 3 subjects receive their assigned dose of picoplatin and leucovorin and 5-FU according to the following schedule:

Day 1: Picoplatin, assigned dosage, as a 2-hour infusion, given either every cycle of 5-FU and leucovorin (q 2 weeks, Schedule A) or with every other cycle of 5-FU and leucovorin (q 4 weeks, Schedule B). Leucovorin, 400 mg/m² in D5W (water-5% dextrose), will be administered as a 2 hour infusion, either alone or, if the patient is to receive picoplatin, at the same time as picoplatin in separate bags using a Y-line. The leucovorin (±picoplatin) will be followed by a 5-FU bolus=400 mg/m² and then by 5-FU, 2,400 mg/m² in D5W administered as a 46 hour continuous infusion.

Subjects in Phase 1 are centrally assigned to one of two schedules of picoplatin. The first cohort of q 2 week (Schedule A) subjects are treated with picoplatin at a dosage of 45 mg/m², every cycle, q 2 weeks. Subsequent sequential cohorts of subjects assigned to this schedule receive picoplatin at dose levels increasing by 15 mg/m² if treatment is well tolerated and until unacceptable dose-limiting toxicity (DLT) establishes the MTD.

The MTD is defined as the dose of picoplatin below the dose at which at least one third of at least 6 subjects experience a DLT. Tolerance data from only the first 4 weeks of treatment is used to determine the MTD. Thus, data following the first two doses of picoplatin in the q 2 week (Schedule A) subjects and following only the first dose of picoplatin in the q 4 week (Schedule B) subjects are considered. The first cohort of q 4 week (Schedule B) subjects will be treated with picoplatin at a dosage of 60 mg/m², every other cycle, q 4 weeks. Subsequent sequential cohorts of subjects assigned to this schedule will receive picoplatin at dose levels increasing by 30 mg/m² if treatment is well tolerated and until unacceptable dose-limiting toxicity (DLT) establishes the MTD. Depending on the pattern and severity of toxicity observed, additional intermediate dose levels of either schedule of picoplatin administration may be studied.

Within each schedule, the cohort size is 3 subjects, and is expanded to 6 subjects if a DLT is observed. Within each cohort of each schedule, one patient is treated initially; if no DLT is observed within the following 4 weeks (2 drug cycles), the remaining two subjects may be treated. If a DLT is observed in the first patient within a cohort, whether or not to proceed with enrollment of additional subjects in the cohort will be determined on a case-by-case basis. All subjects within a q 2 week (Schedule A) cohort will have completed 2 cycles (a cycle=the 2-day treatment regimen and an additional 12-day follow-up period) prior to escalating the dose in the next cohort of subjects. All subjects within a q 4 week (Schedule B) cohort will have completed 1 cycle of the 2-day treatment regimen (which should include 5FU/leucovorin) and an additional 26-day follow-up period prior to escalating the dose in the next cohort of Schedule B subjects.

If no DLT is observed among the 3 subjects within a cohort, picoplatin dose escalation may proceed in the next cohort of that schedule of picoplatin. If one DLT is observed, the cohort size at the specified dose and schedule of picoplatin is expanded to 6 subjects. Additional subjects may be entered at any dosage level and schedule below the dose at which 2 of 6 have DLT to obtain additional safety or efficacy data.

### Phase 2

The dose of the Phase 2 component of this study is selected based on the dose intensity of picoplatin achieved on each dose and schedule, the number of cycles tolerated and a subjective assessment of the tolerability and safety profile of each dose and schedule and a preliminary assessment of response rate in accord with Phase 1. The schedule for Phase 2 is selected as Schedule B, the q 4 week schedule. The subjects (approximately 100 with metastatic CRC, at about 25 clinical sites) are randomized to the modified FOLFOX 6⁶ or to FOLPI-150.

The FOLPI regimen is as follows:

Picoplatin 150 mg/m², is administered with every alternate cycle of 5-FU and leucovorin (q 4 weeks, Schedule B) as a 2 hour infusion. Leucovorin (400 mg/m² in D5W) is administered every 2 weeks as a 2-hour infusion, either alone, or given at the same time as the picoplatin in a separate bag using a Y-line. The administration of leucovorin±picoplatin is followed by a 5-FU bolus of 400 mg/m² and then by 5-FU, 2400 mg/m² in D5W administered as a 46 hour continuous infusion.

The modified FOLFOX 6 regimen is as follows:

Oxaliplatin 85 mg/m², as a 2-hour infusion is administered every 2 weeks. Leucovorin (400 mg/m² in D5W) is administered every 2 weeks as a 2-hour infusion. Oxaliplatin is given at the same time as the leucovorin in a separate bag using a Y-line. The administration of leucovorin+oxaliplatin is followed by a 5-FU bolus of 400 mg/m² and then by 5-FU, 2400 mg/m² in D5W administered as a 46 hour continuous infusion.

Neuropathy assessment is performed at baseline and after every two cycles of therapy (approximately every month) by an independent neurologist. The subject and the neurologist are not informed whether the platinum infused is oxaliplatin or picoplatin. This assessment by the neurologist is used to determine the incidence of Grade 2 or greater peripheral neuropathy. In Phase 2, for the purpose of determining toxicity for dose reduction or study drug discontinuation, the treating physician performs a neurological assessment using the NCI CTCAE. These CTCAE criteria are used to determine the need to dose reduce prior to each cycle. The assessment of the neurologist is used for determination of the safety endpoint, the incidence of neuropathy, and is performed independently every other cycle using the protocol-specified neuropathy scale, but is not be used for dose modification. For all subjects, hematology and serum chemistry laboratory studies are obtained prior to each treatment cycle. Treatment cycles (5-FU and leucovorin±picoplatin or oxaliplatin depending on schedule) are repeated every 2 weeks, but may be delayed up to 2 weeks while awaiting recovery of clinical or laboratory abnormalities. Data from all cycles of treatment and cumulative toxicity are assessed for safety analysis.

Tumor evaluations will be done at baseline and after every 4th treatment of 5-FU/leucovorin (every 8 weeks, unless doses have been delayed) on study. The efficacy endpoint will include objective response rate according to RECIST criteria.²⁶ Duration of response, time to progression, progression-free survival, and overall survival are also evaluated.

The study treatments are summarized in Table 1, below:

TABLE 1
Phase 1 (Dose Escalation)

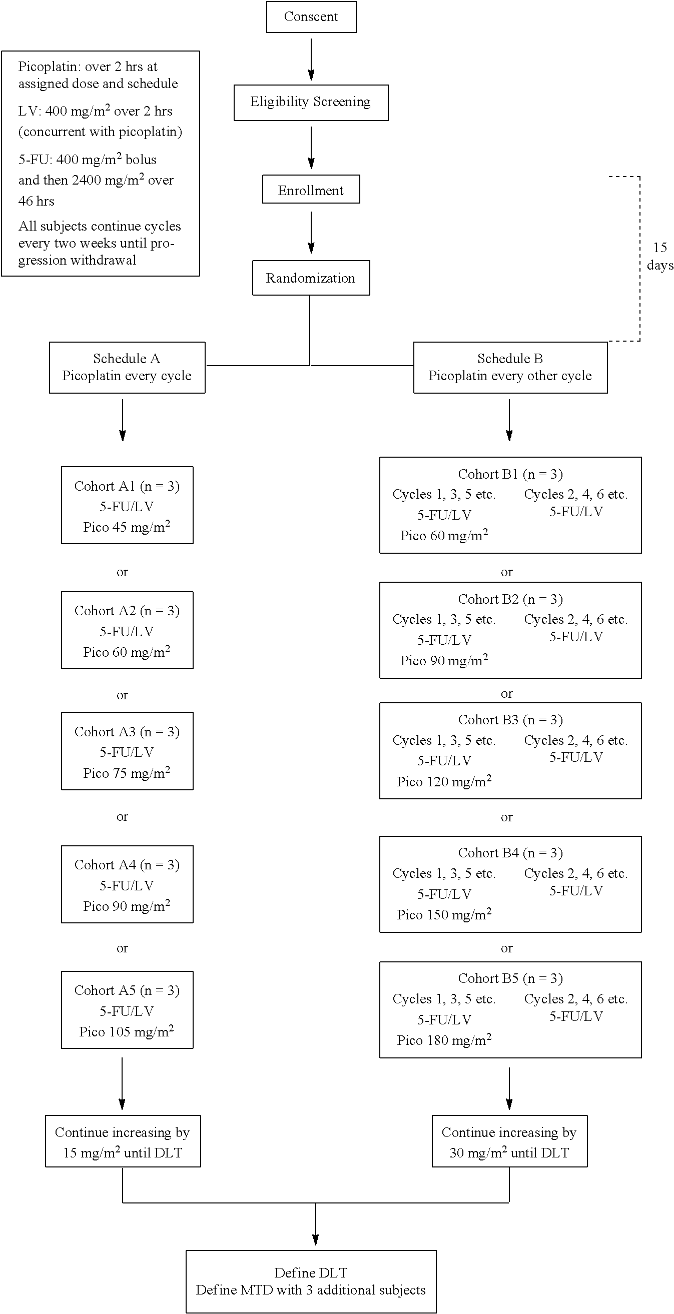

Phase 2

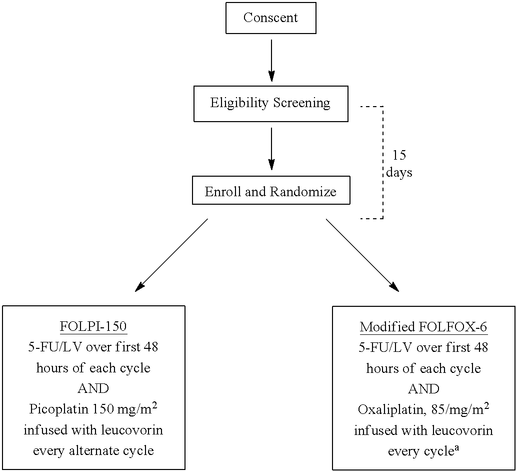

^{a}Picoplatin: over 2 hours 150 mg/m²; oxaliplatin: 85 mg/m², over 2 hours; LV: 400 mg/m² over 2 hours (concurrent with picoplatin when
given or oxaliplatin) followed by 5-FU: 400 mg/m² bolus and then 2400 mg/m2 over 46 hours. All subjects continue cycles every two
weeks until progression or discontinuation of study drug due to toxicity.

### Selection of Picoplatin Dose

Picoplatin was generally tolerated in combination with other myelosuppressive chemotherapeutic agents in previous Phase I studies at doses of 120-150 mg/m² administered every 3 weeks, i.e., doses equivalent to 80-100 mg/m² every 2 weeks or 160-200 mg/m² administered every 4 weeks. None of these studies, however, studied picoplatin in combination with 5-FU and leucovorin. 5-FU/leucovorin is not generally myelotoxic and thus the doses of picoplatin selected as the initial starting doses in the dose escalation portions of the current study, i.e., 45 mg/m² every two weeks and 60 mg/m² every four weeks, were well below the expected MTDs of picoplatin administered on these schedules.

### Administration of Picoplatin

Investigational-site staff must use standard cytotoxic handling procedures when preparing picoplatin for administration. Picoplatin is supplied as a ready-to-use formulation. The contents of the vials must be transferred to a suitable bag for administration. The compatibility of the formulation with typical infusion equipment has been assessed, and results have established compatibility with EVA infusion bags, PVC infusion tubing, and polypropylene syringes when the materials are protected from light. PVC infusion bags are not recommended for administration of picoplatin.

The compatibility of the formulation with typical administration sets has been assessed, and limits of acceptability have been set as 8 hours in a covered infusion bag. The product is highly sensitive to light and should not be exposed to ambient light for more than 1 hour without light protection. The bag must be protected from light during preparation and administration at the time of use.

There is no preservative or bacteriostatic agent present in the picoplatin formulation. Therefore, picoplatin must be transferred under aseptic conditions. The solution must be completely used or discarded within 8 hours of introduction into an infusion bag. As with all platinum complexes, contact with aluminum should be avoided.

Picoplatin should be administered by peripheral vein or central line; it must not be given by the intramuscular or subcutaneous route. The starting dose will be calculated based on the body surface area from the height and weight of the patient. If the patient's weight changes by more than 10%, the treating physician must recalculate the body surface area and amend the dose.

Picoplatin should be administered over 2 hours. It should be administered concurrently with leucovorin, in separate bags using a Y-line, when the two drugs are to be given on the same day. These two drugs have been tested and shown to be compatible when administered in this manner.

Subjects also received anti-emetic therapy consisting of a 5-HT₃ receptor antagonist plus dexamethasone 30 minutes prior to a dose of picoplatin. Subjects may also receive anti-emetic therapy for several days following treatment, which may include oral lorazepam, prochlorperazine, or equivalent for up to 7 days, as clinically indicated for breakthrough nausea and/or vomiting.

### Guidance for Administration

Detailed guidance for administration of 5-FU and leucovorin are provided in the product labels. Briefly, leucovorin 400 mg/m² IV infusion in D5W will be administered over 2 hours at the same time as picoplatin (if picoplatin is to be given on that day), in separate bags using a Y-line, followed by a bolus of 5-FU=400 mg/m² and then by 5-FU 2,400 mg/m² in D5W (recommended) administered as a 46-hour continuous IV infusion.

### Dose Modifications

### Dose Modification of Picoplatin

Dose-reduction is mandatory if any of the following hematological events are observed during the previous cycle: absolute neutrophil count (ANC)<0.5×10⁹/L for at least 5 days; absolute neutrophil count<1.0×10⁹/L complicated with Grade≧2 fever (>38.5° C.); platelet count<25×10⁹/L; not reaching a platelet count≧100×10⁹/L and ANC≧1.5×10⁹/L by Day 15.

Dose reduction is also required for any treatment events involving any treatment-related Grade 3 toxicity, any Grade 4 toxicity, or any renal toxicity or neurotoxicities as described below.

For subjects receiving picoplatin every 2 weeks, the dose reduction should be 15 mg/m²; for subjects receiving picoplatin every 4 weeks the dose reduction should be 30 mg/m².

### Dose Reduction in the Event of Serum Creatinine Changes

Serum creatinine must be measured before every dose of picoplatin. For subjects with abnormal serum creatinine, the dose of picoplatin (but not 5-FU or leucovorin) must be modified according to the following table in Phase 1:

| | Dose modification for | Dose modification for |
|---|---|---|
| Serum Creatinine | q 2 week (Schedule | q 4 week (Schedule B) |
| Value | A) picoplatin subjects | picoplatin subjects |
| ≦institutional ULN | recommended dose | recommended dose |
| >1.0 to 1.5 times ULN | reduce by 25% | reduce by 25% |
| >1.5 to 2.0 times ULN | reduce by 50% | reduce by 50% |
| >2.0 times ULN | discontinue treatment | discontinue treatment |
| | with picoplatin | with picoplatin |

In Phase 2, the following dose reductions will be required for elevated serum creatinine:

| | Dose modification for |
|---|---|
| Serum creatinine | Phase 2 FOLPI subjects |
| ≦institutional ULN | recommended dose |
| >1.0 to 1.5 times ULN | reduce by picoplatin 30 mg/m² |
| >1.5 to 2.0 times ULN | reduce by picoplatin 60 mg/m² |
| >2.0 times ULN | discontinue treatment with |
| | picoplatin |

### Dose Modification in the Event of Neurotoxicity

The dose of picoplatin should be modified according to the CTCAE grade of toxicity and its duration as follows:

| | Duration of Toxicity | |
|---|---|---|
| Toxicity | Resolves before | Persistent |
| Grade | next cycle | (present at start of next cycle) |
| Grade 1 | No change | Maintain picoplatin dose |
| Grade 2 | No change | Reduce picoplatin dose by 30 mg/m² |
| Grade 3 | Reduce picoplatin | Discontinue picoplatin |
| | dose by 30 mg/m² | |

| | |
|---|---|
| Grade 4 | Discontinue picoplatin |

Up to three dose reductions of a 30 mg/m² may occur should toxicity not improve or worsen at a later cycle.

### Dose Modification of 5-FU

The first time the dose of picoplatin is reduced, the bolus dose of 5-FU should be omitted. The second time the dose of picoplatin is reduced, the infusional dose should be reduced by 600 mg/m². Once decreased, the reduced dose of 5-FU should be continued; i.e., the dose of 5-FU should not be subsequently increased.

If the platelet count or ANC count is Grade 1 or 2 at day 15 in a cycle with picoplatin, and the subject receives the alternate i.e., even numbered cycle that does not include picoplatin, the dose of 5-FU should not be reduced at this cycle. At the next treatment cycle, the doses of picoplatin and 5-FU should be reduced by one level. Dose modifications for Grade 3 or 4 non-hematological events must be made. Continue treatment only once toxicity has resolved to <Grade 3.

### Dose Modification of Leucovorin

There are no dose modifications for leucovorin, unless drug sensitivity is suspected because of a temporal relationship to the time of leucovorin administration.

### Results

59 patients have been treated to date in Phase 1. In the q 2 w schedule, 1 of 6 patients showed a DLT of Grade 4 thrombocytopenia and 3 of 6 patients, showed Grade 4 neutropenia at a picoplatin dose level of 105 mg/m². The q 2 w schedule is now being evaluated at 120 mg/m². In the q 4 w schedule, DLT was observed at 180 mg/m² in 2 of 6 patients. The MTD was therefore set at 150 mg/m² in the q 4 w schedule. Patients have received up to 24 cycles and the therapy was well tolerated.

For both schedules, dose delays were primarily from neutropenia or thrombocytopenia, with increased hematological toxicity observed at higher doses. Grade 3 non-hematological toxicities related to treatment include 1 coronary artery spasm following FU infusion, 1 picoplatin infusional allergic reaction, 1 stomatitis, 2 diarrhea, 1 azotemia. The cardiac and stomatitis events were attributed to the 5-FU component. No Grade 2 or higher neuropathy has been reported, even for four patients who have received a cumulative picoplatin dose of greater than about 900 mg/m², a surprising and unexpected result, particularly in view of a high incidence of moderate to severe neuropathy observed at comparable doses of oxaliplatin. This indicates that picoplatin can be safely administered with FU and LV without the dose limiting neuropathy associated with FOLFOX regimens.

In Schedule A (picoplatin q 2 week), the preferred dosage range is about 45-120 mg/m², e.g., doses of 45 to 105 mg/m², e.g., 45 mg/m².

In Schedule B (picoplatin q 4 week), the preferred dose can be higher, e.g., about 120-210 mg/m², e.g., 120-180 mg/m², e.g., 150 mg/m². A lower dose can also be administered, e.g., at 45-90 mg/m², e.g., 60 mg/m².

Of 44 evaluated subjects evaluated by CT scan there have been 6 confirmed partial responses and one complete response (unconfirmed) (16%). Twenty-six of 32 subjects of the Q2 week schedule have been evaluated and 2 partial responses were observed. Surprisingly, 2/3 patients in cohort A1 (45 mg/m²) showed a partial response. Eighteen of 18 subjects in the Q4 week schedule have been evaluated and 5 partial responses were observed (28%).

### Phase 3

The phase 3 study will compare the FOLPI regimen with FOLPI plus bevacizumab, wherein the bevacizumab is administered according to dosing recommendations provided by Genentech for use with FOLFOX regimens in the treatment of MCRC. Tumor evaluations will be done at baseline and after every 4th treatment of 5-FU/leucovorin (every 8 weeks, unless doses have been delayed) on study. The efficacy endpoint will include objective response rate according to RECIST criteria.²⁶ Duration of response, time to progression, progression-free survival, and overall survival will also be evaluated.

### REFERENCES

The following references and other publications, patents and patent applications cited herein are incorporated by reference herein.
1. Jemal et al., Cancer Statistics, 2004. CA Cancer J Clin 54(1): 8-29, 2004.
2. Hoff et al., Oncology (Huntingt) 18(6): 705-708, 2004.
3. Meyerhardt et al., N Engl J Med 352(5): 476-87, 2005.
4. Penland et al., Oncology (Huntingt) 18(6): 715-722, 2004.
5. Saltz et al., N Engl J Med, 343(13): 905-14, 2000.
6. Tournigand et al., J Clin Oncol, 22(2): 229-37, 2004.
7. de Gramont et al., J Clin Oncol, 18(16): 2938-47, 2000.
8. Rothenberg et al., J Clin Oncol, 21(11): 2059-69, 2003.
9. Andre et al., N Engl J Med, 350(23): 2343-51, 2004.
10. Hwang et al., In: Clinical Use of Oxaliplatin: Case Studies and Roundtable Discussion, Editor Marshall J, CMP Healthcare Media, Oncology Publishing Group, Manhasset, N.Y. 2004.
11. Douillard, J Y, Schiller, J., Eur J Cancer 38(Suppl 8): S25-S31, 2002.
12. Beale, P, et al., Br J Cancer 88(7): 1128-1134, 2003.
13. Raynaud F I, et al., Clin Cancer Res 3(11): 2063-2074, 1997.
14. Holford J, et al., Anticancer Drug Des 13(1): 1-18, 1998.
15. Holford J, et al., Br J Cancer 77(3): 366-373, 1998.
16. Rogers P, et al., Eur J Cancer 38(12):1653-1660, 2002.
17. Sharp S Y, et al., Eur J Cancer 38(17):2309-15, 2002.
18. Plasencia C, et al., Invest New Drugs 22(4):399-409, 2004.
19. Murakami H, et al., Eur J Cancer 38(Suppl 8): S1-S5, 2002
20. Giaccone G, et al., Eur J Cancer 38 (Suppl 8): S19-S24, 2002.
21. Gore M E, et al., Eur J Cancer 38(18): 2416-2420, 2002.
22. Treat J, et al., Eur J Cancer 38(Suppl 8): S13-18, 2002.
23. Perez R P, et al., Eur J Cancer 34(10): 1535-42, 1998.
24. Gelmon K A, et al., Ann Oncol 15(7):1115-22, 2004.
25. Gelmon K A, et al., National Cancer Institute of Canada—Clinical Trials Group trial, IND 129. Ann Oncol 14: 543-548, 2003.
26. Therasse P, et al., New Guidelines to Evaluate the Response to Treatment in Solid Tumors. European Organization for Research and Treatment of Cancer, National Cancer Institute of the United States, National Cancer Institute of Canada. J Natl Cancer Inst 92(3): 205-216, 2000.
27. J. M. Tabernero et al., J. Clin. Oncol., 22(145), 3512 (2004).

Useful agents for administration with picoplatin and methods of treatment are also disclosed in include the platinum and non-platinum anticancer drugs disclosed in U.S. patent application Ser. Nos. 10/276,503, filed Sep. 4, 2003; 11/982,841, filed Nov. 5, 2007; 11/935,979, filed Nov. 6, 2007; 11/982,839, filed Nov. 5, 2007; in U.S. Pat. Nos. 7,060,808 and 4,673,668; and in PCT Publication Nos. WO/98/45331 and WO/96/40210.

The following patent applications are incorporated herein by reference in their entireties:

U.S. Ser. No. 61/027,387, filed Feb. 8, 2008, attorney docket no. 295.114prv

U.S. Ser. No. 61/027,382, filed Feb. 8, 2008, attorney docket no. 295.115prv

PCT Ser. No.______, filed Feb. 6, 2009, attorney docket no. 295.115wol

U.S. Ser. No. 61/027,360, filed Feb. 8, 2008, attorney docket no. 295.116prv

PCT Ser. No.______, filed Feb. 6, 2009, attorney docket no. 295.116wol

U.S. Ser. No. 11/982,841, filed Nov. 5, 2007, attorney docket no. 295.093 usl

U.S. Ser. No.______, filed Feb. 6, 2009, attorney docket no. 295.131 usl

1. A method of treatment of colorectal cancer, comprising:
administering to a patient afflicted with colorectal cancer picoplatin, bevacizumab, 5-fluorouracil (5-FU) and leucovorin, wherein 5-FU and leucovorin are administered intravenously at least twice at intervals of about 2-6 weeks, the picoplatin is administered with the leucovorin and 5-FU every other time that the fluorouracil and leucovorin are administered, and the bevacizumab is administered at least twice at one-week or two-week intervals.

2. The method of claim 1, wherein the picoplatin is administered at a dose of about 60-180 mg/m².

3. The method of claim 1, wherein the interval of administration of the 5-FU and the leucovorin is about two weeks and the interval of administration of the picoplatin is about four weeks.

4. (canceled)

5. The method of claim 1 or 3, wherein the picoplatin is administered at a dose of about 45-150 mg/m².

6. (canceled)

7. The method of claim 1 wherein the patient having metastatic colorectal cancer has not previously been treated for metastatic disease.

8. The method of claim 1 wherein the patient having metastatic colorectal cancer has previously been treated with an irinotecan regimen, a FOLFOX regimen, or a FOLPI regimen, wherein the cancer is refractory or wherein the cancer is progressive within six months of completing the regimen.

9. The method of claim 1 wherein the patient having metastatic colorectal cancer has previously been treated successively with at least two of the group consisting of an irinotecan regimen, a FOLFOX regimen and a FOLPI regimen wherein the cancer is refractory or wherein the cancer is progressive within six months of completing the regimen.

10. (canceled)

11. The method of claim 8 wherein the irinotecan regimen or the FOLFOX regimen or both is accompanied by bevacizumab administration.

12. The method of claim 11 wherein the picoplatin and the leucovorin are administered simultaneously.

13. The method of claim 9 wherein the irinotecan regimen or the FOLFOX regimen or both is accompanied by bevacizumab administration.

14. The method of claim 13 wherein the picoplatin and the leucovorin are administered simultaneously.

15. The method of claim 1 wherein the 5-FU is administered following the administration of the picoplatin, leucovorin and bevacizumab.

16. The method of claim 1 wherein the leucovorin and the 5-FU are administered about every two weeks, the picoplatin is administered with the leucovorin about every 4 weeks, and the bevacizumab is administered biweekly.

17. (canceled)

18. The method of claim 1 wherein the leucovorin is administered at an initial dosage of about 200-400 mg/m².

19. The method of claim 1 wherein the 5-FU is administered at a total dosage per dosing of about 1000-3000 mg/m².

20. The method of claim 1 wherein the picoplatin is administered at a dosage of about 120-150 mg/m².

21-25. (canceled)

26. The method of any one of claims 1 or 16 wherein the bevacizumab is administered intravenously at a first dose of about 10 mg/kg, then every other week at a dose of about 10 mg/kg.

27. The method of claim 1 wherein the leucovorin, at a dosage of about 400 mg/m², is administered as a 2 hour infusion, the administration of the leucovorin being followed by a 5-FU bolus at a dosage of about 400 mg/m²; the 5-FU bolus being followed by 5-FU at a dosage of about 2,400 mg/m² administered as a 46 hour continuous infusion; wherein the leucovorin and the 5-FU are administered to the patient every 2 weeks and about 60-150 mg/m² of the picoplatin is administered to the patient with the leucovorin every 4 weeks, wherein at least the initial dose of picoplatin is about 150 mg/m², and wherein the bevacizumab is administered at an initial dose of about 10 mg/kg, then once every other week at a dose of about 10 mg/kg.

28. (canceled)

29. A method of treatment of colorectal cancer comprising:
(a) identifying a patient afflicted with colorectal cancer who has received irinotecan, FOLFOX, or FOLPI regimens, with or without bevacizumab or cetuximab, wherein the cancer is in remission, and
(b) administering about 5-150 mg/m² picoplatin to the patient every 21 days in combination with a dose of about 10 mg/kg bevacizumab, with or without 5-FU or leucovorin or both, administered every other week as an adjuvant therapy to prevent recurrence.

30-65. (canceled)
